(19) United States
(12) Plant Patent Application Publication
(10) Pub. No.: US 2006/0123520 P1
(43) Pub. Date: Jun. 8, 2006

(54) Apple tree named 8S2743

(50) MALUS X DOMESTICA; APPLE

(76) Inventor: W. David Lane, Summerland (CA)

Correspondence Address: Attention: Ken Haddrell;Okanagan Plant Improvement Co. Ltd. (PICO), P.O. Box 6000, Summerland, BC, V0H 1Z0, CA

(21) Appl. No.: 11/003,357

(22) Filed: Dec. 6, 2004

Related U.S. Application Data
(60) Provisional application No. 60/527,286, filed on Dec. 8, 2003.

(51) Int. Cl. A01H 5/00

(52) U.S. Cl. PLT/161

(57) ABSTRACT

The present invention relates to an apple tree and more particularly to a new and distinct variety broadly characterized by its productive and regular-bearing tree that produces fruit that matures under the described conditions in the middle of October at Summerland, British Columbia, Canada. The fruit is large in size, globose conical in shape, with green/yellow skin ground color, high percentage of dark red overcolor, sweet, low acid flavor, and crisp, firm, juicy flesh. The variety was developed from a seedling from a controlled cross between the seed parent ‘Splendour’ and the pollen parent ‘Gala’.

### FILED OF THE INVENTION

Name: ‘8S2743’
Breeders Reference Number 8S-27-43
Genus: Malus
Species: ×domestica
Type: Fruiting apple tree
Market Use: Dessert quality for fresh eating

This invention relates to apple trees and particularly to a seedling apple tree from a controlled pollination done by Dr. W. David Lane of the Pacific Agri-Food Research Centre Summerland apple breeding program located at Summerland, British Columbia, Canada.

The Agriculture and Agri-Food Canada research facility at Summerland was established in 1914. Originally called the Dominion Experimental Farm at Summerland, the name was changed to the Summerland Research Station in 1959, the Summerland Research Centre in 1994 and to the Pacific Agri-Food Research Centre (PARC) Summerland in 1996. The tree fruit breeding program was established in 1924 to provide new varieties for the tree fruit industry of British Columbia, Canada, and the world. The breeding program at Summerland has produced several tree fruit varieties including ‘Spartan’ (unpatented), ‘Silken’ (U.S. Plant Pat. No. 10,740), and ‘Creston’ (U.S. Plant Pat. No. 10,739) apples and ‘Van’ (unpatented), ‘Lapins’ (unpatented), and ‘Sweetheart’ (unpatented) sweet cherries. The tree fruit breeders typically produce several thousand hybrid seedlings each year. Under the direction of the breeder the technician, as part of his duties, propagates the seedlings, by budding onto rootstocks, to induce earlier fruiting, in “seedling” fields. Upon fruiting, the varieties are evaluated for appearance, taste, and texture. Harvest timing, disease susceptibility and growth habit are evaluated in the field. Promising seedlings are re-propagated, in replications of four to six trees, by budding or grafting onto rootstocks and planted out as second selections in randomized evaluation plots complete with reference varieties (commercial varieties). Upon fruiting, selections are evaluated for varietal stability, tree quality, and productivity in the field, and for taste, texture and appearance of the fruit, in “in-house” sensory evaluation panels. The new varieties are compared to reference varieties to establish uniqueness.

The present invention relates to a new and distinct variety of apple tree which was designated ‘8S2743’ in 2001. The original seedling was produced in 1981 and is the offspring of the seed parent ‘Splendour’ (non-patented) and the pollen parent ‘Gala’ (non-patented).
Origin

‘8S2743’ resulted from a cross between the seed parent ‘Splendour’ and the pollen parent ‘Gala’ made at the Pacific Agri-Food Research Centre (PARC), Summerland, BC, Canada, by Dr. W. David Lane (See pedigree). Seedlings from this cross were planted in the greenhouse in early 1981, and transplanted to the field nursery in spring of the same year. The seedling was propagated by budding in place onto Malling 26 EMLA (unpatented) rootstock in the field in 1983. ‘8S2743’ was selected on the basis of fruit quality and tree habit in 1989, assigned the breeder's number 8S-27-43, and propagated for second test on Malling 26 EMLA beginning in 1990. The second test trees were planted in randomized, replicated trials in the spring of 1992. In 1993, additional trees were propagated by budding onto Malling 9 (unpatented) rootstock and planted in a randomized replicated trial with standard cultivars as comparators. All propagated trees were evaluated from the time of first fruiting for yield, fruit size, fruit quality, storage and sensory quality over 10 years with consistent results. In 1997, the selection was forwarded to elite stage testing under the designation 8S-27-43.
History of the Invention

The present invention relates to a new and distinct variety of apple tree which was named ‘8S2743’ in 2001. The controlled cross was made in 1980 by Dr. W. David Lane. Seeds from fruit of the cross were collected at fruit maturity and isolated from seeds from other crosses. The seeds were subjected to the required moist chilling treatment (stratification) to enable the seeds to break dormancy in the fall of 1980. After the required stratification the seeds were germinated in a greenhouse. The resulting seedlings were transplanted into pots and grown in a greenhouse in the spring of 1981. The seedlings were taken from the greenhouse in May 1981 and planted in the field nursery at the PARC Summerland. Propagative wood from the seedling was taken and budded in place onto Malling 26 (unpatented) rootstock in 1983. Malling 26 is used to hasten the onset of fruiting and dwarf the tree. This particular seedling was budded at tree position 43 in row 27 in a field designated 8S. The seedling subsequently fruited and observations determined the fruit had unique qualities of possible commercial potential. The seedling was given the Breeder's Reference Number 8S-27-43 in 1989. Four trees were created by T-budding vegetative buds onto Malling 26 rootstock in August of 1990. The resulting trees were planted in replicated, randomized trial block at the PARC Summerland. Five more trees were made by budding onto Malling 9 rootstock in August of 1993. The second test trees were planted in replicated trials with standard cultivars and evaluated from the time of first fruiting for harvest timing, fruit size, fruit quality, storage and sensory quality over 10 years with consistent results. In 1997, the selection was forwarded to elite stage testing under the designation 8S-27-43.

Controlled grower trials, under test agreements, have been established in British Columbia, and selected research sites.

### SUMMARY OF THE INVENTION

Trees of ‘8S2743’ produce fruit that is harvested mid to late in the harvest season, and has bi-colored skin (dark red overcolor and green/yellow ground color). The apple is a dessert apple (Malus×domestics Borkh.) with exceptional texture and keeping quality. The fruit flesh is crisp and juicy, firm, and sweet. It retains crispness and juiciness for 5 months in regular air storage at 1° C. The shelf life of fruit after storage is excellent. The tree is highly productive. The growth habit of trees of ‘8S2743’ is upright/spreading with ample spur development and moderate vigor. It is not subject to alternate bearing or pre-harvest drop.

Trees of ‘8S2743’ have been tested, under a testing agreement, by several orchardists in BC, Canada, and at selected research sites.

### BRIEF DESCRIPTION OF THE DRAWINGS

The accompanying photographic sheets show various characteristics of the apple. variety ‘8S2743’. The colors of the photographs are as nearly true as is possible in a color representation of this type.

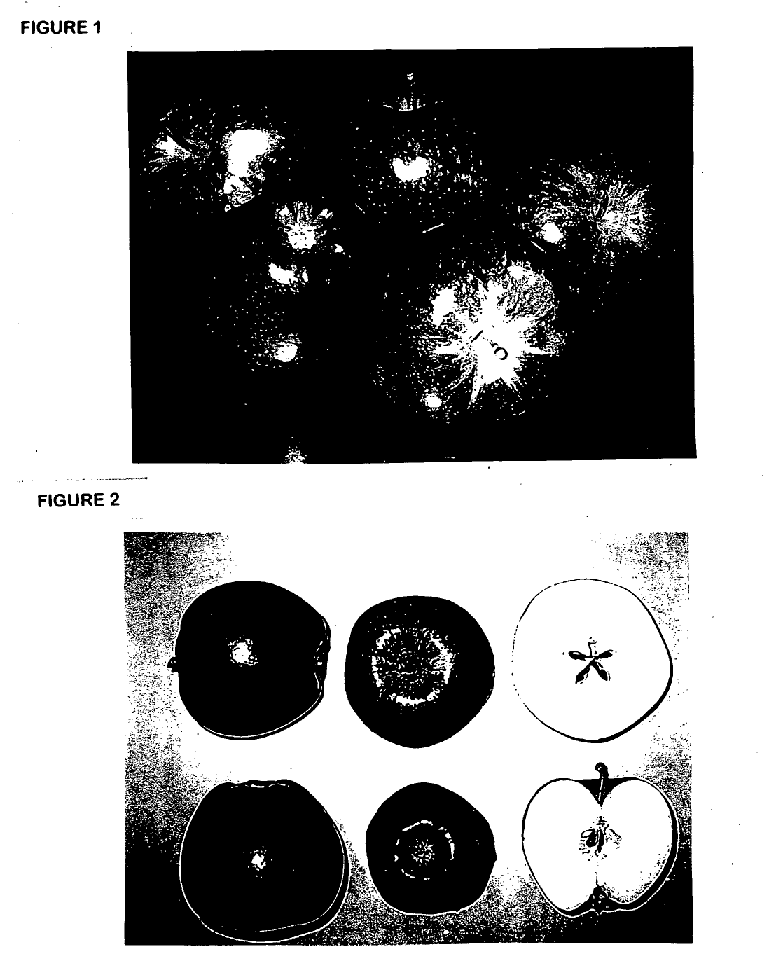

In photograph sheet I, FIG. 1, typical fruit are shown. The fruit are displayed in various views to show the globose conical shape in the side view, and views of the basal and calyx ends which show some degree of russetting.

In photograph sheet I, FIG. 2, 2 typical apples are shown. The photograph also shows the fruit cut transversally at the core line and longitudinally. These views expose the core, carpels, and also show the eye basin and calyx. The fruit shown are close to optimum maturity. The fruit are displayed to show the top and basal views of the fruit.

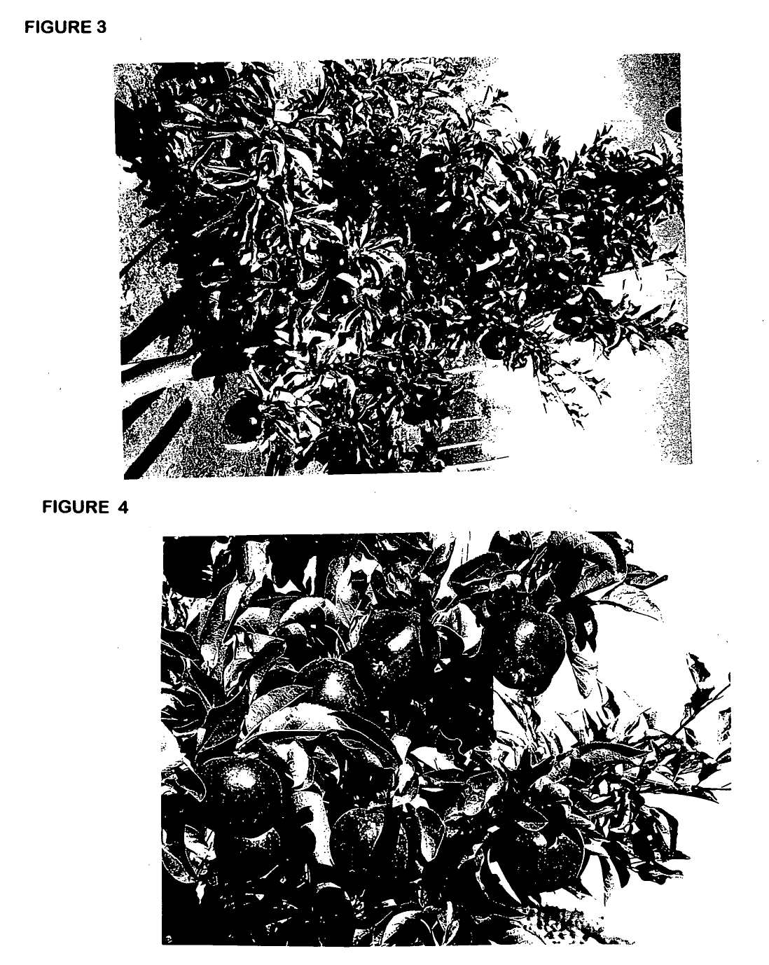

In Photographic Sheet II, FIG. 3, a typical tree is shown. The photograph shows fruit on the tree at about harvest maturity.

In Photographic Sheet II, FIG. 4, typical fruit on branches are shown. The photograph shows the heavy cropping ability of the variety.

### DISTINGUISHING CHARACTERISTICS

Under growing conditions at the Pacific Agri-Food Research Centre (PARC) Summerland located at Summerland in the Okanagan Valley of British Columbia, Canada, the variety ‘8S2743’ consistently has the following characteristics that distinguish it from other apple varieties. The trees are moderately vigorous, spurry, and have wide branch angles. The tree is very productive and regular bearing. Cumulative yield of ‘8S2743’ after 6 cropping years averaged 77.2 kg per tree on Malling 26 rootstock, significantly more productive than ‘Braeburn’ (unpatented) at 42.9 kg per tree, or ‘Golden Delicious’ (unpatented) at 50.4 kg per tree. On Malling 9 rootstock, cumulative yield after 5 years was 75.6 kg per tree for ‘8S2743’, 40.6 kg per tree for ‘Braeburn’, 53.1 kg per tree for ‘Fuji’ (unpatented) and 57.7 kg per tree for ‘Royal Gala’ (patent expired).

Trees of ‘8S2743’ are moderately vigorous. The trunk girth after 7 years for trees propagated on Malling 26 rootstock averaged 19.0 square cm, significantly larger than 10.9 sq. cm for ‘Braeburn’ and 12.4 sq. cm for ‘Golden Delicious’. (Trunk girth is highly correlated with aboveground tree size.) On Malling 9 rootstock after 5 years, trunk girth of ‘8S2743’ was 22.5 sq. cm, similar to ‘Fuji’ (21.0 sq. cm) and larger than the other standards in the plot (‘Fuji’, ‘Gala’, ‘Braeburn’, ‘Jonagold’ (unpatented)). In controlled freeze tests, the tree of ‘8S2743’ was similar to ‘Golden Delicious’ in winter hardiness.

The fruit of ‘8S2743’ are large and globose-conical in shape. Fruit size estimated in the field by counting and weighing all the fruit on the tree, averaged 267 g over 6 years for ‘8S2743’ on Malling 26 rootstock, compared to 246 g for ‘Golden Delicious’. On Malling 9 rootstock, fruit size of ‘8S2743’ was 319 g over 5 years, compared to 275 g for ‘Fuji’ and 225 g for ‘Royal Gala’. Skin color is 90% dark red over a yellow-green ground color. Color change begins well in advance of harvest maturity. The fruit have medium stem bowl russetting and low skin russetting. Russet is more severe in cool, wet conditions. There is some russetting on the lenticels. The stem bowl russetting does not usually break over the shoulders of the fruit. The fruit can be harvested in one or two picks. The average date of harvest over 8 years was October 11 (day 285) (Table 1).

Analytical laboratory measurements of the fruit flesh firmness, soluble solids, starch index and titratable acidity are shown in Table 1. Firmness at harvest averaged 19.8 lb, soluble solids 13.5%, and titratable acidity 0.5%. The flesh of the fruit is firm, very crisp, and juicy, and the taste is sweet and very low in acid. Trained judges assessed the attributes of ‘8S2743’ fruit relative to ‘Fuji’ (unpatented) in 8 panels over 6 years, ranging from 47 to 92 days after harvest of ‘8S2743’. Fruit of ‘8S2743’ were judged to have tougher skin than ‘Fuji’, flesh that was crisper, harder and less juicy than ‘Fuji’, and flavor that was sweeter and less sour than ‘Fuji’, but no different in flavor intensity (Table 2).

The fruit have an exceptionally long storage life, at least 5 months in air at 1° to 2° C., and a long shelf life. Firmness measurements at harvest, after air storage, and after a 7-day shelf life test appear in Table 3. Although some firmness is lost over the course of these tests, on Day 7 of the shelf life test, the fruit firmness still exceeded 15 lb in all but one instance (Table 3).

The variety was derived from a seed from the cross of the seed parent ‘Splendour’ (unpatented) and pollen parent ‘Gala’ (unpatented) made at the Agriculture and Agri-Food Canada, Pacific Agri-Food Research Centre, Summerland B.C. in 1980.

Plant material from the seedling cross was propagated, by budding on M 26 (unpatented) rootstock, on the 43rd tree, in row 27, in field 8S, at the Pacific Agri-Food Research Centre (PARC) Summerland in 1983. The resulting tree was given the Breeder's Reference Number 8S-2743. Evaluation on the selection began upon fruiting. 8S-27-43 was evaluated for fruit appearance, taste, flesh texture, tree quality, and productivity. 8S-27-43 proved to be a tree that was very productive and produced fruit of high quality with exceptionally long storage life. The variety was selected for further evaluation and 4 trees were made, by budding on Malling 26 rootstock, and planted out in a randomized, replicated trial block at the PARC Summerland in 1992. The variety has been propagated on Malling 9 and Malling 26 rootstocks and planted out at the PARC and, under test agreements, at other sites. All trees resulting from propagation by budding or grafting have been true-to-type with no variants or off types occurring. The variety is stable and unique.
Pomolgical Charateristics of the Apple Tree Named ‘8S2743’
Flower:
   Color of bud at full balloon stage.—Pink/White.
   Flower type.—Single.
   Fragrance.—Fragrant.
Fruit:
   Shape.—Globose-Conical.
   Shape in cross-section.—Regular.
   Ribbing.—Present but minimal.
   Side view.—Asymmetric.
   Size.—Large.
   Length.—78 mm.
   Diameter.—87 mm.
   Average fruit weight at harvest.—267 g to 319 g.
Skin Color:
   Ground color at full maturity.—150D RHS Yellow-Green.
   Ground Color after storage.—150D RHS Yellow-Green.
   Overcolor.—53A RHS Dark Red, approx. 90% of fruit surface
   Blush.—Present.
   Skin thickness.—Medium.
   Skin glossiness.—Medium.
   Russet.—Present.
   Position of russet.—Stem bowl and lenticels.
   Bloom.—Present.
Lenticels:
   Lenticel color.—White.
   Position of lenticels.—Cheeks.
   Lenticel russet.—Present.
   Stem length.—30 mm.
   Stem thickness.—3.4 mm.
Cavity:
   Depth.—23 mm.
   Width.—39 mm.
   Surface.—Smooth.
Eye basin:
   Depth.—13 mm.
   Width.—33 mm.
   Shape.—Round.
   Rim.—Slight Crowning.
Calyx:
   Sepals.—7mm.
   Curvature.—Upright to recurved.
   Shape.—Short to medium and narrow.
   Aperture of eye.—Open.
   Eye size.—Medium.
   Calyx tube.—Urn-Shaped.
   Stamens.—Median.
   Core-lines.—Meeting.
   Core.—Basal.
   Size.—Medium.
Carpels:
   Shape in cross section.—Open.
   Shape in longitudinal section.—Mucronate.
   Symmetry.—Symmetric.
   Inner surface.—Slight tufting.
   Seed number.—Seven.
   Shape.—Conical.
   Seed testa.—None.
   Color at maturity.—Brown.
Flesh:
   Texture.—Crisp.
   Juiciness.—Juicy.
   Graininess.—None.
   Flesh browning.—medium to strong.
   Firmness at harvest.—19.8 lbs.
   Flavor.—Slightly aromatic, sweet, low acidity.
   Titratable acidity.—0.5%.
   Soluble solids.—13.5%.
   Flesh Color.—11D Yellow.
   Maturity season.—Second week of October, one week after Red Delicious.
   Number of picks.—1-2.
   Storage.—105-121 days in air storage.
   Shelf life.—Longer than Red Delicious, similar to Fuji.
   Use.—Eating, cooking, dessert.
Tree: Trees of ‘8S2743’ have been tested on M.26 EMLA and M.9 EMLA (unpatented) rootstocks with slender spindle training system.
   Vigor.—moderate on M26 and M9.
   Shape.—Upright spreading.
   Spurs.—Spurry.
   Bearing habit.—Spurs.
   Bearing Efficiency.—Heavy annual crops.
   Pre-harvest fruit drop.—Negligible.
   Precocity.—Medium.
   Crop.—Annual.
   Branch angle.—Wide.
   Productivity.—Very Good.
   Thinning.—Moderate.
Dormant first-year shoots:
   Internodes at middle third of shoot.—31 mm.
   Lenticel color.—White.
   Lenticel size.—Moderately small.
   Density.—Medium to high (Avg. 141 over 12 cm in center of shoot).
   Bark color on sunny side.—1887A RHS.
Two-year-old wood:
   Bark color on sunny side.—166A RHS.
   Lenticels.—Medium number.
   Lenticel shape.—Round to a Horizontal Oval.
   Lenticel color.—White.
   Lenticel size.—Medium.
   Hardiness.—6A. Similar to ‘Golden Delicious’ in artificial freeze tests
Leaves:
   Growing tip color.—Green.
   Cross sectional shape.—Concave.
   Pubescence on upper side.—Weak.
   Abaxial surface color.—147A.
   Anthocyanin coloration.—None.
Mid-shoot fully expanded leaves:
   Orientation.—Upright to Slightly Outward.
   Blade width.—63 mm.
   Blade length.—107 mm.
   Blade length.—width ratio 1.7.
   Apex shape.—Acuminate.
   Leaf margins.—Serrate.
   Pubescence on abaxial side.—Weak.
   Anthocyanin coloration of veins.—Strong.
   Petiole length.—Average of 27 mm.

Stipule length.—Average of 8.4 mm.

TABLE 1
Laboratory assessment of fruit quality 8S-27-43 at harvest in different
years and different fields. Values are the means of 10-fruit samples, unless
otherwise noted.

| | | Date | | | | Titratable |
|---|---|---|---|---|---|---|
| | | (day | | | Soluble | acidity |
| Harvest | Harvest | of | Firmness | Starch | solids | (% maleic |
| Year | Field | year) | (pounds) | index^{z} | (%) | acid) |
| 1997 | A | 281 | 18.9 | 3.0 | 14.5 | 0.6 |
| | A | 289 | 18.2 | 3.2 | 13.1 | 0.5 |
| | A | 295 | 19.1 | 3.6 | 12.9 | 0.5 |
| 1998 | A | 261 | 20.4 | 6.0 | 13.6 | 0.6 |
| | A | 266 | 20.0 | 6.6 | 13.4 | 0.5 |
| 1999 | A | 282 | 19.8 | 4.6 | 12.1 | 0.5 |
| | B | 281 | 20.9 | 3.4 | 12.0 | n/d |
| 2000 | A | 276 | 21.1 | 4.4 | 12.8 | 0.6 |
| | B | 277 | 20.8 | 3.2 | 13.2 | 0.6 |
| 2001 | A | 297 | 18.6 | 4.0 | n/d | n/d |
| | A | 300 | 21.0 | 2.6 | 13.7 | 0.6 |
| | B | 305 | 18.9 | 4.0 | n/d | n/d |
| | C | 300 | 19.3 | 2.8 | n/d | n/d |
| 2002 | A | 290 | 20.2 | 3.8^{y} | 14.4 | 0.6 |
| | B | 290 | 19.2 | 4.3^{y} | n/d | n/d |
| | C | 289 | 20.0 | 4.6^{y} | 14.5 | 0.5 |
| 2003 | C | 280 | 19.1 | 5.9^{y} | 14.6 | 0.5 |
| 2004 | C | 273 | 21.6 | 3.8^{y} | 13.5 | 0.5 |

n/d no data available
^{z}Starch index was rated on 5 fruit using the generic 1 to 8 scale of Blanpied and Silsby (1992)
^{y}Mean of 10 fruit

TABLE 2

| | Skin | | | | Flavor | | |
|---|---|---|---|---|---|---|---|
| | tough- | Crisp- | Hard- | Juici- | Inten- | Sweet- | Sour- |
| Selection | ness | ness | ness | ness | sity | ness | ness |
| 8S-27-43 | 5.7 | 6.8 | 7.3 | 5.8 | 4.5 | 5.6 | 3.2 |
| Fuji | 5.4 | 6.4 | 6.8 | 6 | 4.4 | 5.4 | 3.8 |

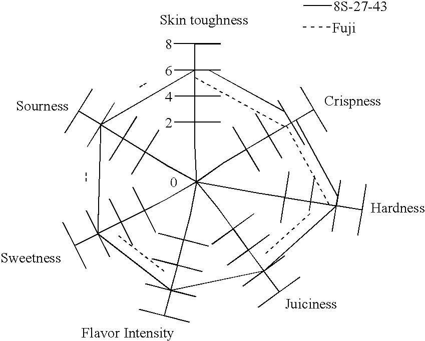

TABLE 3
Shelf life test for 8S-27-43 in different years. The fruit
were removed from 1° C. air storage, and the firmness measured (Day 0).
After one week at 20° C., the firmness was measured again.

| | Harvest | | | Day 0 | | |
|---|---|---|---|---|---|---|
| | date | | | date | | |
| | (day | Harvest | | (day | Day 0 | Day 7 |
| Harvest | of | firmness^{z} | Days in | of | firmness^{z} | firmness^{y} |
| Year | year) | (lb) | storage | year) | (lb) | (lb) |
| 1995 | 273 | nr | 117 | 32 | 18.5 | 16.7 |
| 1995 | 280 | nr | 123 | 31 | 13.9 | 14.7 |
| 1996 | 289 | nr | 117 | 41 | 17.2 | 17.2 |
| 1997 | 295 | 19.1 | 98 | 28 | 16.7 | 16.3 |
| 1998 | 261 | 20.4 | 130 | 26 | 19.2 | 17.3 |
| 1998 | 261 | 20.4 | 144 | 40 | 17.6 | 17.7 |
| 1998 | 266 | 20.0 | 125 | 26 | 17.1 | 15.9 |
| 1999 | 281 | 20.9 | 118 | 34 | 18.6 | 18.3 |
| 2002 | 290 | 20.2 | 99 | 24 | 18.1 | 19.2 |

^{z}Mean of 10 fruit
^{y}Mean of 5 fruit. The same fruit were tasted in hedonic panels on Day 7
nr = not recorded

Pedigree of the Apple Tree named 8S2743

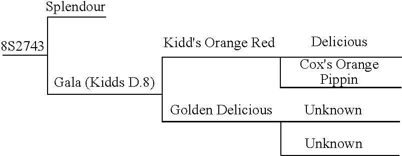

We claim:

1. A new and distinct variety of apple tree, originating from a controlled cross of ‘Splendour’×‘Gala’ substantially illustrated and described, characterized as to novelty by the harvest timing, green/yellow skin ground color, high percentage of dark red overcolor, bright luster, globose conical shape, long storage and shelf life, low fruit acidity, high tree productivity and the unique combination of fruit taste, texture and juiciness which are different from other dessert quality apples.
